(19) United States
(12) Patent Application Publication
(10) Pub. No.: US 2026/0335532 A1
(43) Pub. Date: Oct. 29, 2026

(54) IRRIGATION DEVICE WITH USER CUSTOMIZABLE RETENTION

(71) Applicant: Hollister Incorporated, Libertyville, IL (US)

(72) Inventors: Jerome A. Henry, Castlebar (IE); William K. Arnold, Gurnee, IL (US)

(21) Appl. No.: 19/487,269

(22) PCT Filed: May 23, 2024
(86) PCT No.: PCT/US2024/030718

§ 371 Date: Nov. 24, 2025

Related U.S. Application Data
(60) Provisional application No. 63/505,168, filed on May 31, 2023.

(51) Int. Cl. A61M 3/02

(52) U.S. Cl. CPC A61M 3/0202; A61M 3/0258; A61M 3/0295; A61M 2205/52; A61M 2210/1064; A61M 2210/1067

(57) ABSTRACT

An irrigation device including a catheter with a retention balloon and a controller. More particularly, an irrigation device with a controller configured to incrementally adjust the inflation size of the retention balloon. More particularly, an irrigation device with a controller configured to remember an adjusted inflation size of a retention balloon.

The present application claims the benefit of and priority to U.S.

Provisional Application No. 63/505,168, filed May 31, 2023, which is hereby incorporated by reference.

### FIELD OF THE DISCLOSURE

The present disclosure is directed to trans-anal irrigation (TAI) and stoma irrigation devices, methods and systems for antegrade irrigation such as colostomy/stoma irrigation. More particularly, the present disclosure is directed to an irrigation device that has a controller configured to allow the user to vary the inflation of the balloon to a desired comfort level, wherein the controller remembers the level of inflation for future irrigation procedures.

### BACKGROUND

Many individuals suffering spinal cord injury (SCI) and other medical conditions (e.g., cauda equina syndrome, multiple sclerosis (MS), spina bifida (SB), and chronic constipation) may need to avail themselves of bowel management treatments, in many cases along with a bladder management program. For SCI users, the issues of independence, dexterity, and ease of use are important needs that must be addressed by a bowel management program. Users can avail themselves of various solutions such as pharmacological (laxatives/suppository), digital stimulation, diet control and others, with the aim of having a regular bowel management routine without constipation or fecal incontinence.

Trans-anal irrigation (TAI) provides another option for bowel management. TAI is the delivery of irrigating liquid into the colon to flush the system of stool and create pseudo-continence for the end user. Systems currently on the market allow the user to utilize a product over the toilet, in a commode/shower chair or in a bed to introduce water into the bowel through a rectal catheter. The user will introduce an amount of water into the bowel (typically 500-700 mL) in order to flush out stool located in the bowel passage. The user will typically introduce the water, wait for a period of time and allow gravity to flush the water and stool out of the body.

Generally, the rectal catheter may have an inflatable/deflatable balloon to assist in retention of the catheter during water introduction. The balloon is typically inflated by a fluid such as air or water. Inflation may occur via a manual or automatic means. For example, in some irrigation devices, the retention balloon may be inflated by a manual hand pump. The retention balloon volume may be determined by how many times the user squeezes a hand pump. By inflating the balloon manually, it is possible to rupture the balloon if the pump is squeezed too often. Additionally, such hand pumps may be tiresome to use, resulting in hand fatigue. Furthermore, the balloon may be underinflated, resulting in premature leakage of irrigant and effluent, which may result in an incomplete rectal irrigation.

In other examples, an irrigation device may automatically inflate a retention balloon. Typically, in these instances, the device may provide a number of pre-determined inflation sizes to prevent the balloon from being overinflated and rupturing. However, pre-determined sizes may not accommodate everyone. For instance, a pre-determined size may be too large for a user, thus causing them discomfort or a pre-determined size may be too small for a user, thus leading to premature leakage.

Therefore, an improved irrigation device, system, and/or method allowing a user to adjust the inflation of the retention balloon is needed.

### SUMMARY

In one aspect, the present disclosure is directed to an irrigation device including a pump, an irrigation fluid reservoir in fluid communication with the pump, and a rectal or stoma catheter in fluid communication with the pump and the irrigation fluid reservoir. The rectal or stoma catheter includes a retention balloon.

The irrigation device also includes a controller configured to inflate the retention balloon to a pre-determined default inflation size, incrementally adjust the inflation size of the balloon from the pre-determined default inflation size, and remember the adjusted balloon inflation size.

In another aspect, the present disclosure is directed to a method for trans-anal or stoma irrigation. The method includes inserting a catheter of an irrigation device into the rectum or stoma. The catheter includes a retention balloon. Additionally, the irrigation device includes a pump in fluid communication with the catheter, an irrigation fluid reservoir in fluid communication with the pump and the catheter, and a controller. The controller is configured to inflate the retention balloon to a pre-determined default inflation size, incrementally adjust the inflation size of the retention balloon from the pre-determined default inflation size, and remember the adjusted inflation size of the retention balloon. The method further includes inflating the retention balloon to the pre-determined default inflation size, irrigating the rectum or stoma, deflating the retention balloon, and removing the catheter.

### BRIEF DESCRIPTION OF THE DRAWINGS

FIG. 1 is a perspective view of an example of an irrigation device of the present disclosure in a condition ready for use.

FIG. 2 is a perspective view, on an enlarged scale, of the catheter and a portion of the dual-lumen tubing shown in FIG. 1.

FIG. 3 is a perspective view of an example of a pump base unit of the irrigation device shown in FIG. 1.

FIG. 4 is a front view of one embodiment of a controller display screen of one embodiment of the controller of the irrigation device shown in FIG. 1 during Stage 1, the inflation of the retention balloon.

FIG. 5 is a diagrammatic view of the hydraulic control circuit during Stage 1, the inflation of the retention balloon.

FIG. 6 is a front view of the controller display screen of the controller of the irrigation device shown in FIG. 1 during Stage 2, the flowing of irrigation fluid.

FIG. 7 is a diagrammatic view of the hydraulic control circuit during Stage 2, the flowing of irrigation fluid.

FIG. 8 is a front view of the controller display screen of the controller of the irrigation device shown in FIG. 1 during Stage 3, the deflation of the retention balloon.

FIG. 9 is a diagrammatic view of the hydraulic control circuit during Stage 3, the deflation of the retention balloon.

FIG. 10 is a diagrammatic view of the hydraulic control circuit during a power fault condition.

FIG. 11 is a perspective view of an alternate example of an irrigation device of the present disclosure in a condition ready for use.

FIG. 12 is a perspective view of an example of a pump base unit of the example of the irrigation device shown in FIG. 11.

FIG. 13 is a diagrammatic view of the hydraulic control circuit, showing connections to a connector hub and a portion of the rectal catheter.

FIG. 14 is a front view of the controller display screen of the controller of the irrigation device shown in FIG. 11 with all of the stage icons showing for illustrative purposes.

FIG. 15 is a front view of the controller display screen of the controller of the irrigation device shown in FIG. 11 during Stage 1, the priming of the waste control valve and the catheter.

FIG. 16 is a diagrammatic view of the hydraulic control circuit during the first phase of Stage 1, the priming of the waste control valve.

FIG. 17 is a diagrammatic view of the hydraulic control circuit during the second phase of Stage 1, the priming of the catheter.

FIG. 18 is a front view of the controller display screen of the controller of the irrigation device shown in FIG. 11 during Stage 2, the inflation of the retention balloon.

FIG. 19 is a diagrammatic view of the hydraulic control circuit during Stage 2, the inflation of the retention balloon.

FIG. 20 is a perspective view of an example of a catheter of the irrigation device shown in FIG. 11 with the retention balloon inflated with water.

FIG. 21 is a front view of the controller display screen of the controller of the irrigation device shown in FIG. 11 during Stage 3, during which irrigation fluid flows through the catheter into the user's rectum.

FIG. 22 is a diagrammatic view of the hydraulic control circuit during Stage 3, during which irrigation fluid flows through the catheter into the user's rectum.

FIG. 23 is a perspective view of an example of a catheter of the irrigation device shown in FIG. 11 during Stage 3, indicating the flow of irrigation fluid from the patient-proximal end of the catheter.

FIG. 24 is a front view of the controller display screen of the controller of the irrigation device shown in FIG. 11 during Stage 4, during which liquified waste fecal matter exits through the catheter openings and main passage into a toilet or waste collection bag.

FIG. 25 illustrates how the seven-segment display on the controller display screen indicates the state of the waste control valve during Stage 4; one or the other of these displays would appear, not both, indicating whether the waste control valve is open or closed.

FIG. 26 is a diagrammatic view of the hydraulic control circuit during Stage 4.

FIG. 27 is a perspective view of the catheter of the irrigation device shown in FIG. 11 during Stage 4, indicating the flow of liquified waste fecal matter into the patient-proximal end of the catheter and out of the patient-distal end of the catheter.

FIG. 28 is a front view of the controller display screen of the controller of the irrigation device shown in FIG. 11 during Stage 5, the deflation of the retention balloon.

FIG. 29 is a diagrammatic view of the hydraulic control circuit during Stage 5.

FIG. 30 is a perspective view of the catheter of the irrigation device shown in FIG. 11 after Stage 5, showing the deflated balloon.

### DETAILED DESCRIPTION OF THE EMBODIMENTS

A more detailed description of the device in accordance with the present disclosure is set forth below. It should be understood that the description of the specific devices below is intended to be exemplary, and not exhaustive of all possible variations or applications. Thus, the scope of the disclosure is not intended to be limiting and should be understood to encompass variations or embodiments that would occur to persons of ordinary skill.

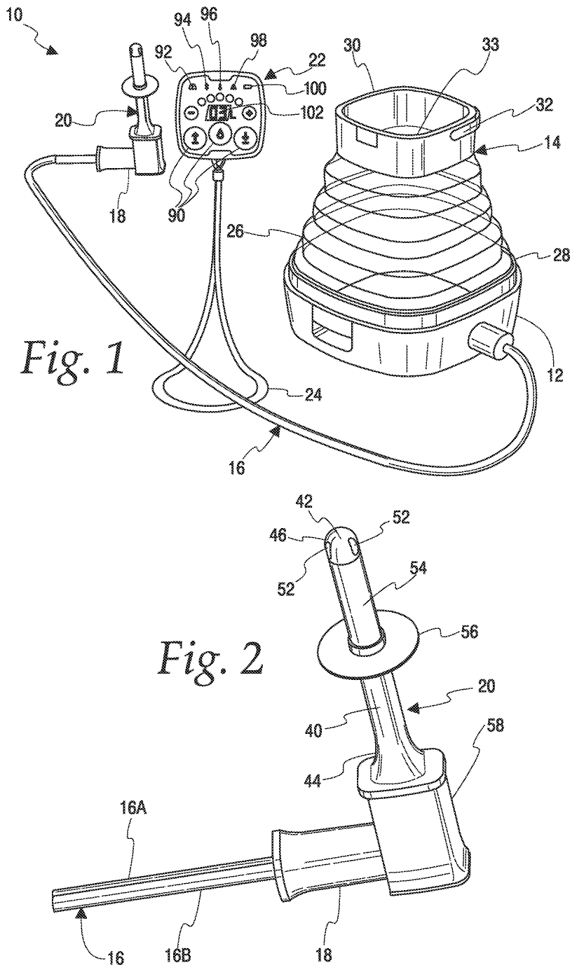

The present disclosure is directed to an irrigation device, such as a trans-anal irrigation (TAI) or stoma irrigation device, which is shown generally at 10 in FIG. 1 where it is shown deployed for use. It will be understood that when the term “irrigation device” is used herein that this term is inclusive of a stoma irrigation device, unless otherwise stated. The main components of the device 10 include a pump base unit 12, an irrigation fluid reservoir 14, fluid tubing 16, a tubing connector 18, a disposable catheter 20 (which may be a rectal or stoma catheter), and a controller 22, with an optional lanyard 24 attached to the controller. Although controller 22 is illustrated and described as a wireless controller, controller 22 may be otherwise operatively associated with the device, such as by a wire.

The reservoir 14 may have a flexible side wall 26 that extends from an upstanding outer wall 28 at the bottom to a collar 30 at the top. The flexible side wall 26 may have a corrugated construction. For example, the side wall 26 may be formed by three step sections of progressively smaller outer dimension from bottom to top. A more detailed disclosure of the reservoir 14, including side wall 26 is disclosed in International Application Publication WO 2018/009871, which is hereby incorporated by reference herein in its entirety.

The collar 30 may have a handle 32 pivotably connected to it. A user can pivot the handle up 90° from the position illustrated in FIG. 1 to carry the reservoir 14. The collar 30 defines an opening 33 at the top of the reservoir. This opening may receive a funnel (not shown) therein. The funnel may have a fill tube connected to it. The funnel can be removed from the collar 30 and placed underneath a faucet for filling the reservoir. The free end of the fill tube would be placed through the collar 30 and into the reservoir cavity for this purpose. Water from the faucet flows through the funnel and fill tube and into the reservoir 14.

It will be understood that if the reservoir 14 is removable from the pump base unit 12 then the reservoir bottom wall will have a valve in it that provides selectable fluid communication between the interior of the reservoir and a conduit joined to one of the pump flow control valves. The valve automatically closes when the reservoir 14 is removed from the pump base unit 12 and automatically opens when the reservoir is mounted on the pump base unit 12. The pump base unit may also mount a temperature sensor (not shown in FIG. 1) that electronically communicates with the controller 22.

External features of the catheter 20 are shown in FIG. 2. The rectal catheter has a shaft 40 which for most of its length is generally cylindrical with two separate passages, a balloon passage 17 (shown in FIG. 5) and an irrigant passage 19 (shown in FIG. 7), therethrough. The shaft 40 has a patient-proximal end 42 and a patient-distal end 44. The patient-proximal end terminates at a rounded tip 46 with a plurality of apertures 52 therein. The apertures 52 provide fluid communication with the irrigant passage in the interior of the shaft 40. A retention balloon 54 (shown in FIG. 2 in the deflated state) is mounted on the exterior of the catheter shaft 40 at a location near the patient-proximal end 42. The patient-proximal end of the catheter, including the deflated retention balloon 54, will be inserted into the rectum during a TAI procedure. An optional inflationless cuff 56 is shown below the retention balloon. The cuff 56 is made of a soft, spongy material (such as silicone but other materials could be used) and may be inserted into the rectum to help retain the catheter and prevent leakage.

The patient-distal end 44 of the catheter shaft 40 flares outwardly to join a catheter hub 58. The catheter hub 58 is joined to a tubing connector 18, establishing fluid communication between the tubing 16 and the catheter 20. The fluid tubing 16 includes an irrigant tube 16A and a balloon tube 16B, as best seen in FIG. 2. These two tubes define lumens therein which are entirely separate from one another and do not fluidly communicate at any point. A detailed disclosure of the inner construction of the connector 18 and catheter hub 58 is provided in International Application Publication WO 2018/009871, which has been incorporated herein by reference in its entirety, above.

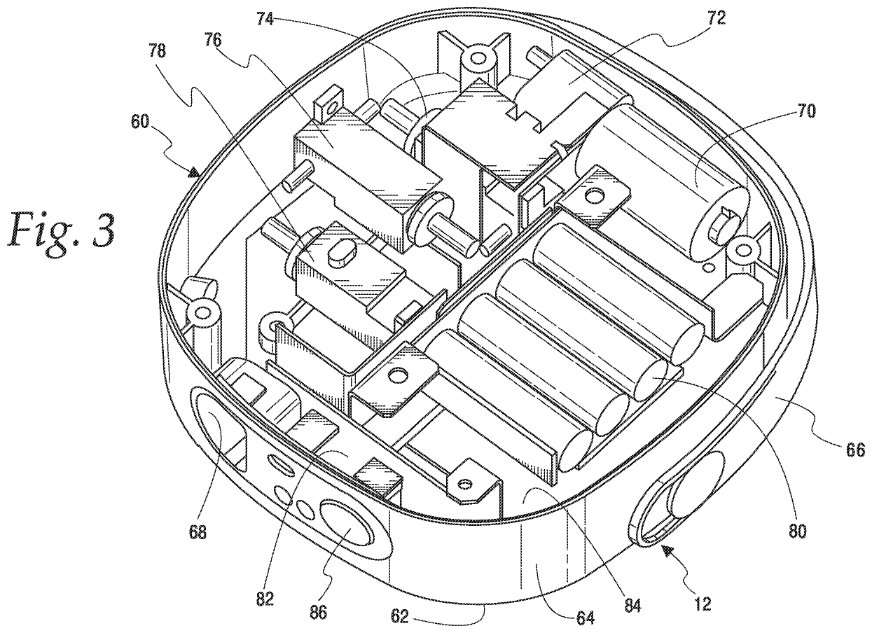

Turning now to the pump base unit 12, FIG. 3 illustrates details of the pump base unit 12. It has a generally hollow shell 60 which includes a floor 62 and a perimeter wall 64. The wall 64 supports the base plate of the reservoir 14 when the reservoir is installed on the pump base unit 12. The wall 64 has a handle 66 pivotably connected to it. A user can pivot the handle up 90° from the position illustrated in FIG. 3 to carry the pump base unit 12. The wall 64 also has an opening 68 through it for mounting a fitting on the end of the tubing 16. The fitting allows connection of the tubing 16 and provides fluid communication between the pump and the fluid tubing.

Inside the shell 60 there is an electric motor 70, a one-way pump 72 and three solenoid valves. The solenoid valves include a reservoir flow director valve 74, a pump flow director valve 76, and a tubing flow director valve 78. The solenoid valves are normally-open, three-way valves. Also present within the shell 60 is a rechargeable battery pack 80 for powering the pump 72 and internal tubing (not shown in FIG. 3 for clarity) which provides various fluid connections among the solenoid valves 74-78, the reservoir conduit and the pump 72 and the tubing 16.

The fluid connections provided by the internal tubing are described below in the fluid circuit diagrams. A power circuit board 82 and a controller printed circuit board 84 are also in the shell 60. A power button 86 on the outside of the shell turns the pump base unit on and off.

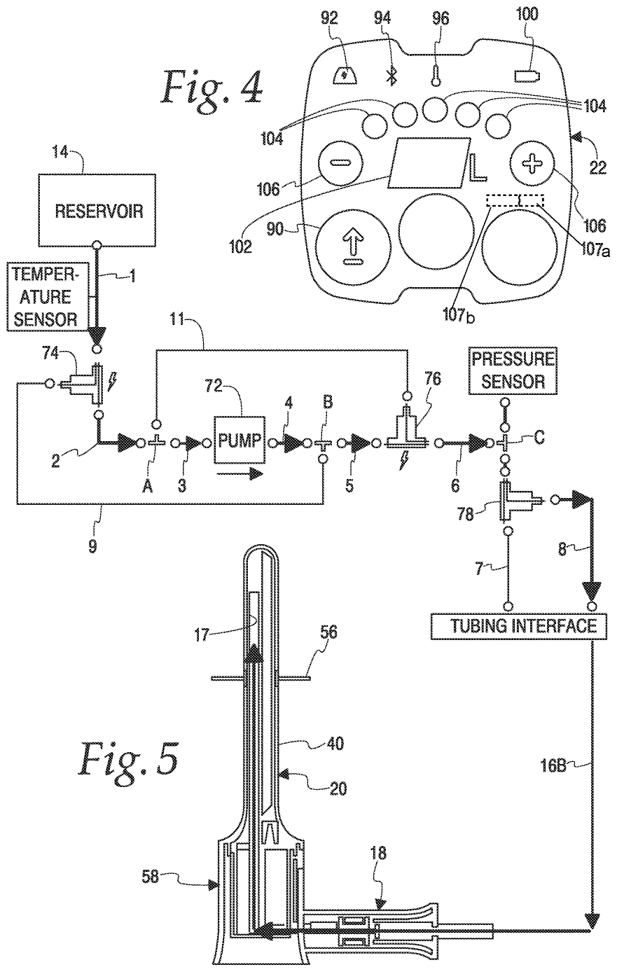

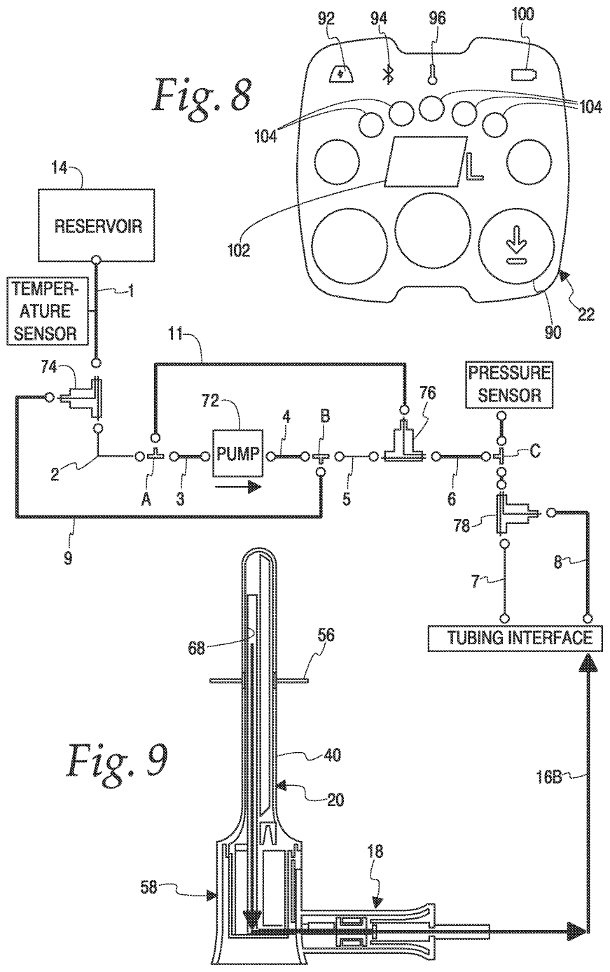

FIGS. 1, 4, 6, and 8 show details of the controller 22, which is illustrated as a wireless controller but may be operatively connected to the device by any suitable connection. The controller has its own rechargeable battery. The display includes stage icons in a row across the bottom of the display as shown at 90. A brief description of the stages and icons is as follows. Stage 1 is the inflation of the retention balloon. The Stage 1 icon is an upwardly pointing arrowhead. Stage 2 is the introduction of irrigation fluid. The Stage 2 icon is a water droplet. Stage 3 is the deflation of the retention balloon preparatory to withdrawal of the catheter. The Stage 3 icon is similar to the Stage 1 icon but with downwardly pointing arrowhead. Other icons may be used to depict the stages without departing from the scope of the disclosure.

Across the top of the controller display, there is a battery power indicator 92 for the pump base unit, a Bluetooth icon 94 for indicating communication between the controller 22 and the pump base unit 12, a water temperature gauge 96, a fault indictor 98 (which lights only if there is a problem) and a controller battery power indicator 100. The battery power indicators will continuously display the battery power level. A green color indicates that the battery has adequate power to complete a TAI procedure, whereas a red color advises the user that the battery should be recharged. A seven-segment display 102 can be used to indicate the amount of liquid pumped either to the balloon or to the catheter apertures. Depending on the stage selected, the numeric display may also show the percentage of stage completion, or other information relevant to the current activity.

A series of five circles 104 and the seven-segment display 102 can be used during Stage 1 to indicate the amount of balloon inflation. The retention balloon sizes, one to five, may be pre-defined so that they are identical for all users. For example, balloon size one may be 9 mm, size two may be 27 mm, size three may be 45 mm, size four may be 63 mm, and size five may be 80 mm. The inflated balloon size should not exceed 80 mm, and the controller may be programmed to not inflate the balloon beyond 80 mm. A practitioner may advise each individual user what predetermined retention balloon size they should select, which will be automatically remembered for their next irrigation procedure, i.e., there is a programming mode via the controller. Additionally, the catheter 20 may include an inherent pressure relief valve to prevent over-inflation of the retention balloon within the rectum.

A user may also adjust the balloon size during the irrigation procedure if needed by using the + (plus) and − (minus) buttons 106, e.g. if leakage occurs after water is instilled into the rectum. For instance, the controller may be configured to adjust the balloon volume by introducing or removing water in increments. In some embodiments, the increments may be as small as 10 mL. The controller may be configured to adjust in balloon volume in other increments without departing from the scope of the disclosure.

The controller display could provide feedback to quantify the incremental increase/decrease through either a decimal or percentage output. For example, the display 102 may display values of 3.05, 3.1, 3.7, etc., to indicate that the balloon size is between the predetermined balloon sizes three and four. In some embodiments, the + (plus) and − (minus) buttons 106 may display the same color as the stage button and the circles. For example, when the upwardly pointing arrowhead is displayed to indicate inflation of the balloon, the plus and minus buttons will display the same colors. Additionally, the controller is configured to automatically remember any adjustments the user made to be able to automatically inflate to the adjusted balloon size during subsequent use. For example, the controller includes a processor 107a and a memory 107b where adjusted inflation values may be stored.

FIGS. 5, 7, 9, and 10 illustrates one example of a hydraulic control circuit that may be employed with the controllers described herein. It will be understood that the hydraulic control circuit is being described for the purpose of illustrating the features of the controllers described and the controllers described herein may be used with other irrigation devices and hydraulic control circuits that operate in different manners. In addition to the items previously described, the hydraulic control circuit includes a reservoir conduit 1 providing fluid communication between the reservoir 14 and the reservoir flow director valve 74. The reservoir flow director valve 74 is further connected to a reservoir outlet conduit 2 and a T-connector A, which is itself connected to a pump inlet conduit 3. Conduit 3 joins pump 72. Pump outlet conduit 4 connects to T-connector B. Return line 9 joins T-connector B to reservoir flow director valve 74. T-connector B also joins a further conduit 5 which supplies the pump flow director valve 76. A reservoir recirculation conduit 11 joins pump flow director valve 76 to T-connector A. An alternate outlet of valve 76 is conduit 6 which goes to T-connector C. A pressure sensor may be connected to T-connector C, as is the tubing flow director valve 78. Valve 78 connects to both conduit 7, which is irrigant tube 16A, and to conduit 8, which is balloon tube 16B.

The use, operation and function of the irrigation device 10 and its hydraulic control circuit are as follows and is being provided as one example of a device that could be used with the controllers according the present disclosure. In preparation for use the user unpacks the irrigation device as shown in FIG. 1. The controller 22 is removed from its storage location in the collar 30 of the reservoir.

The reservoir 14 is filled either by removing it from the pump base unit 12 and carrying it to a faucet, or by removing a funnel from the reservoir and placing the funnel under a faucet with the fill tube extending from the funnel to the reservoir.

Either way, the reservoir 14 is filled with warm tap water (or other source of water such as, but not limited to, bottled water), at the appropriate temperature (between 28 °C and 38 °C) and placed back on the pump base unit 12 if need be. One end of the tubing connector is inserted into the opening 68 of the pump base unit 12 and the other end is attached to the catheter hub 58. Note that the pump base unit 12 is not powered on during this preparation phase, although it could be.

The next step is to power up the electronic controller 22 and the pump base unit 12. The base unit will undergo a system self-check prior to enabling operation. Bluetooth wireless pairing will be established between the remote controller and the base unit when the controller 22 is wireless. As shown in FIG. 3, the base unit contains three 3-way solenoid valves 74, 76, 78 and a unidirectional pump 72 which provide fluid pathways to the catheter for irrigation and to/from the retention balloon. Note that fluid pathways can be established by other embodiments, i.e. reversible pump or dual pumps in combination with 2-way or 3-way solenoid valves. Each of the solenoid valves will only be energized at its appropriate stage and for the duration required, so as to prevent fluid back pressure and noise. A pressure sensor is utilized to monitor fluid path pressure. The electronic control of the fluid pathways, pressure sensor interface, communication with the wireless Bluetooth remote controller, and battery pack charging are provided by two printed circuit board assemblies, namely the power pcb 82 and main controller pcb 84.

The unit will perform a water temperature check via the temperature sensor and indicate on the remote controller interface what range the temperature falls within. The water temperature will be indicated from the thermometer icon which will have three LED's; a blue color indicates the water temperature is <28 °C, a green color indicates the water temperature is between 28 °C-38 °C while a red color indicates the water temperature is >38 °C (in this latter instance, the pump will not operate).

In the following description of the hydraulic control circuit, passageways that are closed by one of the solenoid valves 74-78 are shown in a light weight line, while the heavy lines indicate where liquid is flowing. Arrows indicate the direction of active flow.

The following description is provided in reference to TAI. However, it will be understood that the same or similar process may be carried out for stoma irrigation.

### Stage 1: Rectal Catheter Retention Balloon Inflation

The rectal catheter 20 is safely inserted into the rectum, to its correct position as per the clinician training. Only the retention balloon inflation stage button, the +and -buttons 106, seven segment display 102, and circles 104 will alight on this occasion on the wireless controller 22. When the user wishes to inflate the retention balloon 54 to the size indicated on the controller interface, the solenoid valves 74 and 76 in FIG. 5 are energized by pressing the balloon inflation stage icon button. The green circles 104 will incrementally alight to the selected balloon size (one to five, left-to-right), to provide feedback to the user that the stage is in progress. Should the user wish to adjust the inflation volume of the balloon 54, they may press the + or − buttons 106 to increase or decrease the inflation volume. The graphical display 102 will indicate any adjustments made to the inflation volume.

Should the user wish to pause inflating the retention balloon 54 at any time during their TAI procedure, they simply press the retention balloon inflation stage icon button. After the retention balloon has been inflated to the pre-determined or adjusted size, the user may press the instill irrigant stage icon to advance to the next stage, or the controller may automatically progress onto the next stage (instill irrigant). In instances where the inflation size of the balloon is adjusted from a pre-determined default size during Stage 1, the controller 22 is configured to remember/store the adjusted size for subsequent use. For example, processor 107a and memory 107b will remember/store the adjusted size for subsequent use so that during Stage 1 of a subsequent TAI procedure with device 10, the retention balloon 54 will be inflated to the adjusted size of the previous TAI procedure.

When the retention balloon stage is activated, FIG. 5 illustrates how water from the reservoir shall travel through the lumen to the retention balloon, via the three solenoid valves and pump; the tubing path sequence is 1-2-3-4-5-6-8. Water increases the balloon volume, thus retaining the catheter inside the rectum.

### Stage Two: Transfer of Irrigant From the Reservoir Into the Rectum

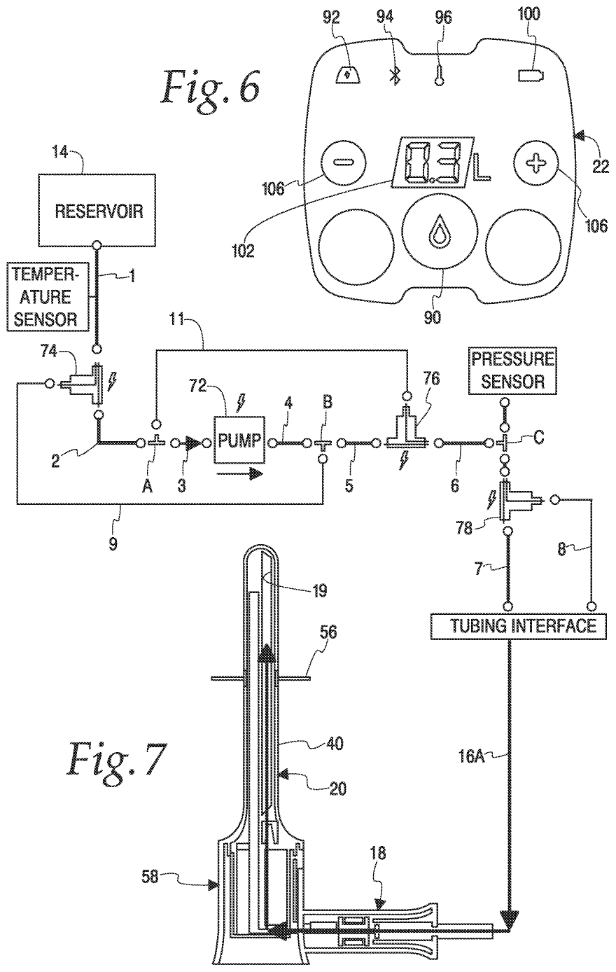

The next step for the user is to instill irrigant from the water reservoir into the rectum. Only the instill water stage icon button, + and − buttons 106 and seven segment display 102 will alight on this occasion, as shown in FIG. 6. The seven segment display 102 will indicate the volume of irrigant to be inserted; the volume can be adjusted in 100 mL increments using the + and − buttons 106. It will be observed that the + and − buttons 106 now have the same color as the instill irrigant stage button and the seven segment display 102.

Per FIG. 7, water from the reservoir 14 shall travel through the separate lumen for the catheter apertures 52, via the three solenoid valves 74, 76, 78 all energized and pump 72; the tubing path sequence is 1-2-3-4-5-6-7. Consequently, water will pass through the irrigant tube 16A, through the irrigant passage 19, and out through the tip apertures 52 and irrigate the rectum. Once the required amount of irrigant has been pumped, the pump 72 will turn off and the controller 22 will progress onto the retention balloon deflation stage.

Should the user wish to pause instilling irrigant at any time during their TAI procedure, they simply press the instill irrigant stage icon button.

### Stage Three: Rectal Catheter Retention Balloon Deflation

After the appropriate volume of water has been inserted into the rectum, the user will then wish to remove the rectal catheter 20 from their rectum. Only the retention balloon deflation stage icon button will be lit up, along with the number of circles 104 corresponding to the balloon size and/or the display 102 indicating any adjustments made to the pre-determined balloon size. The + or − buttons 106 will not be available for the user to select in this instance either. The user simply presses the stage button to activate this phase of the TAI procedure.

As shown in FIG. 9, because the pump is one-way, and not reversible, the tubing path sequence is: 8-6-11-3-4-9-1 (all solenoids are de-energized). Once the retention balloon 54 has been completely deflated, the pump 72 is turned off. The user can then safely remove the catheter 20 from the rectum, disconnect the catheter 20 from the connector 18 and dispose of the catheter 20 hygienically.

### Power Fault Condition

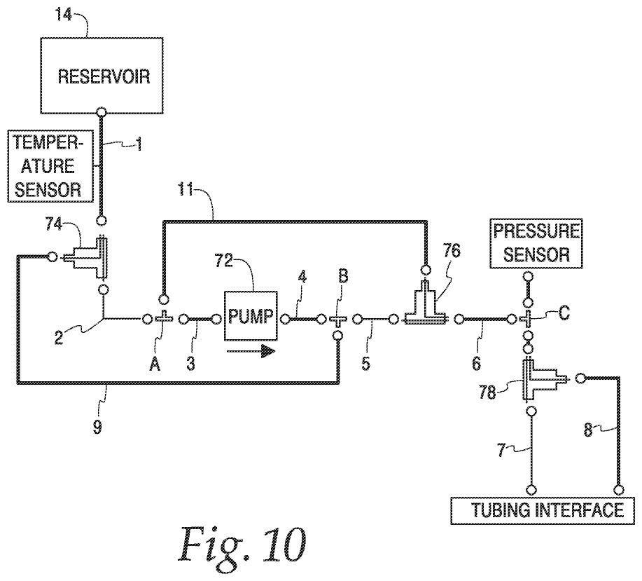

Should there be a power fault while the user is undergoing a TAI procedure, then the pump 72 and all of the solenoid valves will become de-energised. This is to ensure that the retention balloon 54 deflates immediately so that the rectal catheter 20 can be removed safely from the patient's rectum. FIG. 10 shows that the tubing path sequence is: 8-6-11-3-4-9-1.

It will be noted that in this disclosure all lumens are independent to each other, and there is no physical means for them to communicate with each other. Also, the device of the present disclosure ensures that water from the deflated catheter balloon 54 only returns to the water reservoir 14, and not into the catheter's irrigant lumen. Thus, the design ensures that the water does not travel back to the catheter 20 and unnecessarily fill the rectum with the “left over” water from the balloon 54.

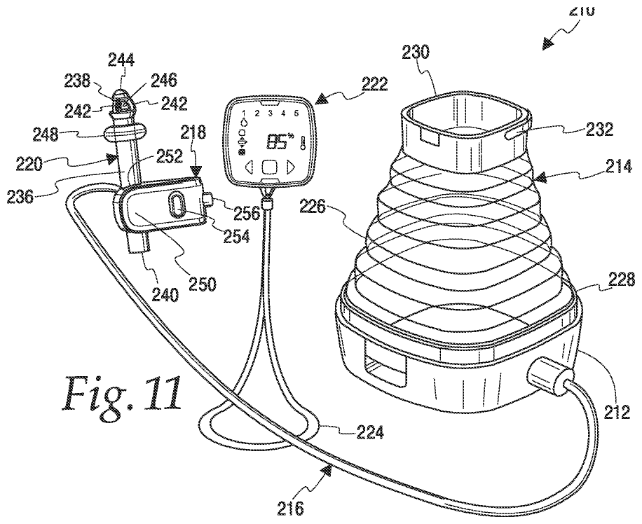

FIG. 11 shows another example of an irrigation device 210 shown deployed for use. Device 210 includes similar features and components as device 10 disclosed herein. The main components of device 210 include a pump base unit 212, an irrigation fluid reservoir 214, fluid tubing 216, a connector hub 218, a disposable catheter 220 (which may be a rectal or stoma catheter), and a controller 222, which may be a wired or wireless controller. The wireless controller may have an optional lanyard 224 attached to it.

The reservoir 214 may be the same as the reservoir described herein. Alternatively, the reservoir 214 may be any of the reservoirs provided in International Application Publication WO 2018/009871, which has been incorporated by reference herein in its entirety, above. The collar 230 may have a handle 232 pivotably connected to it. A user can pivot the handle up 90° from the position illustrated in FIG. 11 to carry the reservoir. The collar defines an opening at the top of the reservoir. This opening may normally receive a funnel therein. The funnel may have a fill tube connected to it.

The base plate 228 includes a projecting tube (not shown) that engages a valve in the pump base unit 212 to provide selectable fluid communication between the interior of the reservoir and a conduit joined to one of the pump flow control valves. The projecting tube engages a valve that automatically closes when the reservoir is removed from the pump base unit 212 and automatically opens when the reservoir is mounted on the pump base unit 212. The base plate may also mount a temperature sensor (not shown) that electronically communicates with the controller 222.

FIG. 11 also illustrates features of the catheter 220. The catheter has a hollow shaft 236 which has a patient-proximal end 238 and a patient-distal end 240. The patient-proximal end 238 includes three vanes 242 which are spaced 120 ° apart from one another. The vanes taper to a rounded tip 244. Adjacent pairs of vanes 242 define an aperture 246 between them. The apertures 246 provide fluid communication with the main passage in the interior of the shaft 236. A retention balloon 248 (shown in FIG. 11 in the inflated state) is mounted on the exterior of the catheter shaft 236 at a location near the patient-proximal end 238. The patient-proximal end of the catheter, including the deflated retention balloon 248, will be inserted into the rectum or stoma during an irrigation procedure.

The connector hub 218 is seen in FIG. 11 in what is generally a front elevation view. In a top plan view the hub 218 has a U-shaped configuration in the nature of a clevis. One of the two arms of the clevis is seen at 250. Together the two arms define a slot at 252 in the hub which releasably receives the catheter shaft 236 and its manifold (the manifold will be described below). A manifold release button, one of which is seen at 254, is located on the front and back of the hub 218. Inside the hub there are front and rear hooks (not shown) which are releasably engageable with the manifold. Pushing the release buttons 254 retracts the hooks which allows removal of the manifold and shaft 236 from the hub's slot 252. Thus, the hub is a reusable part of the irrigation device 210 which is intended for continuing use, while the catheter 220 is a disposable part of the assembly which is intended for only a single, one-time use. The right side of the hub as seen in FIG. 11 has a nipple 256 which is engageable with the fluid tubing 216.

Further details of the catheter 220 are shown in FIG. 13. In this figure the catheter 220 is shown with the shaft 236 truncated such that not all of the patient-proximal end 238 is shown and the retention balloon 248 is shown only diagrammatically by a single line. An optional inflationless cuff 258 is shown below the retention balloon. The cuff 258 is made of a soft, spongy material (such as silicone but other materials could be used) and may be inserted into the rectum or a stoma to help retain the catheter and prevent leakage. The catheter further includes a manifold, indicated diagrammatically at 260, surrounding the shaft 236. The shaft 236 is removably connected to the connector hub 218 by sliding the manifold 260 into and out of the clevis slot 252 in the end of the hub 218.

The shaft 236 defines a main passage 262 that extends through the shaft. Although not shown in FIG. 13, it will be understood that the main passage 262 opens at the top, or patient-proximal end 238, through the apertures 246 between the vanes 242. In this example the shaft 236 itself terminates in the manifold 260 but the main passage 262 continues through the manifold and to a tail piece 264 attached to the bottom of the manifold. Thus, the main passage 262 opens at the bottom at the patient-distal end 240. Inside the manifold there is a waste control valve, shown diagrammatically at 266 in the main passage 262 near the patient-distal end 240 of the tail piece 264. The waste control valve 266 selectably opens and closes the main passage 262 of the catheter 220.

Generally, the internal construction of the manifold 260 includes a balloon passageway 268, a catheter passageway 270 and a control valve passageway 272. A more detailed description of the internal construction of the manifold 260 is provided in International Application WO 2018/009871, which has been incorporated herein by reference in its entirety.

The tubing 216 includes three separate lumens, a catheter lumen 386, a balloon lumen 384, and a waste control valve lumen 388. When connected to the catheter 220, the catheter lumen 386 is in fluid communication with the catheter passageway 270, the balloon lumen 384 is in fluid communication with the balloon passageway 272, and the waste control valve lumen 388 is in fluid communication with the control valve passageway 272. The control valve passageway 272 may also include a one-way valve or check valve 274 to prevent fluid flow from the main passage 262 into the connector hub 218, and thus prevents contamination of the hub 218 or fluid tubing 216 with fecal matter. It will be understood that for clarity of the drawing, the hub 218 is shown only diagrammatically and is not shown surrounding the manifold 260 as in FIG. 11.

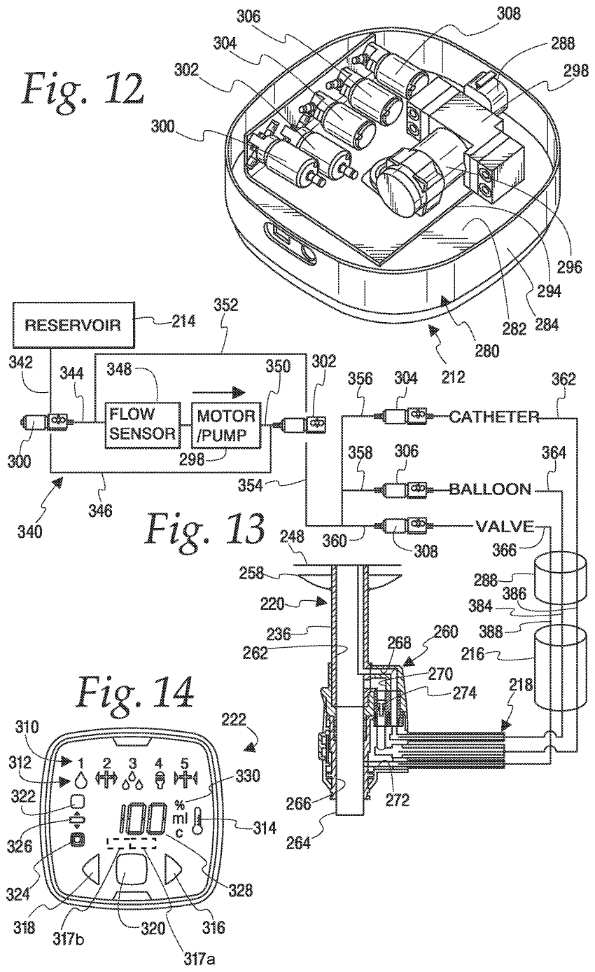

FIG. 12 illustrates details of the pump base unit 212. It has a generally hollow shell 280 which includes a floor 282 and a perimeter wall 284. The wall 284 supports the base plate 228 of the reservoir 214 when the reservoir is installed on the pump base unit 212. The wall 284 may have a handle (not shown) pivotably connected to it allowing a user to conveniently carry the pump base unit 212. The wall 284 also has an opening through it for mounting a fitting 288. The fitting 288 allows connection of the fluid tubing 216 and provides fluid communication between the pump and the fluid tubing.

Looking at FIG. 12, inside the shell 280 there is a mounting plate 294 which supports an electric motor 296, a pump 298 and five solenoid valves. The solenoid valves include a reservoir flow director valve 300, a pump flow director valve 302, a catheter valve 304, a balloon valve 306, and a waste valve 308. Solenoid valves 300 and 302 are normally-open, three-way valves. Solenoid valves 304, 306 and 308 are normally-closed, two-way valves. Not shown but present within the shell 280 are a rechargeable battery for powering the pump 298 and internal tubing which provides various fluid connections among the solenoid valves 300-308, the reservoir conduit, the pump 298 and the fitting 288. The fluid connections provided by the internal tubing are described below in the fluid circuit diagrams.

FIG. 14 shows details of the controller 222, which as illustrated as a wireless controller. The controller has its own rechargeable battery. The display includes stage numbers 1-5 in a row across the top of the display as shown at 310. Beneath each stage number is an icon. The row of icons is indicated at 312. A brief description of the stages and icons is as follows. Stage 1 is the priming of two of the fluid tubing 216 lumens, namely, the catheter lumen 362 and the control valve lumen 368. The Stage 1 icon is a single large water droplet. Stage 2 is the inflation of the retention balloon 248. The Stage 2 icon is a cross with outwardly pointing arrowheads, the cross suggesting a vertical catheter shaft and a transverse balloon. Stage 3 is the introduction of irrigation fluid. The Stage 3 icon is three small water droplets. Stage 4 is opening of the waste control valve 266 and evacuation of waste material from the rectum. The Stage 4 icon represents a toilet. Stage 5 is the deflation of the retention balloon 248 preparatory to withdrawal of the catheter 220. The Stage 5 icon is similar to the Stage 2 icon but with inwardly pointing arrowheads. The icons will display in blue when a stage is selected (all other icons will be turned off). The icon will turn white when the selected stage is activated and the icon will flash when its function is underway. Other icons may be used to depict the stages without departing from the scope of the disclosure.

Continuing from the stage icons 312 clockwise around the perimeter of the controller display, there is a water temperature gauge 314 which may be three LED's, red, green, and blue. Red indicates the reservoir water temperature is too high, blue indicates the water temperature is too low, and green indicates the water temperature is just right.

At the bottom of the display are stage selection buttons including a forward button 316 and a back button 318. Pressing the forward button 316 advances the selected stage number by one while pressing the back button 318 reduces the selected stage number by one. When the desired stage is reached an activate stage button 320 is pressed to cause activation of the selected stage. When the activate stage button 320 is pressed the selected stage's icon will change from blue to flashing white until the stage is completed. Upon completion of the stage the icon will stop flashing and remain white. Additionally, forward button 316 and back button 318 may be used to make adjustments to the system during an irrigation procedure. For example, forward button 316 and back button 318 may be used to adjust the inflation size of the retention balloon 248 from a pre-determined default size. The controller is configured to automatically remember any adjustments the user made to be able to automatically inflate to the adjusted balloon size during subsequent use. For example, the controller includes a processor 317a and a memory 317b where adjusted inflation values may be stored.

Two battery level indicators 322 and 324 are separated by a power on indicator LED 326. Indicator 322 is for the controller battery and indicator 324 is for the pump motor battery. The battery level indicators turn green when full power is available, amber when the available battery power is low, and red when the battery is depleted. A numeric display 328 is located in the middle of the controller. Depending on the stage selected, the numeric display may show the percentage of stage completion, the percentage of balloon inflation, the volume of liquid pumped, the water temperature, whether the waste control valve is open or closed, or other information relevant to the current activity. One of the units indicators 330 may be lit as appropriate. From top to bottom in the illustrated example the units indicators are for percentage, milliliters, and degrees Celsius.

FIG. 13 illustrates the hydraulic control circuit 340 of the present disclosure. In addition to the items previously described, the hydraulic control circuit 340 includes a reservoir conduit 342 providing fluid communication between the reservoir 214 and the reservoir flow director valve 300. The reservoir flow director valve 300 is further connected to a pump inlet conduit 344 and a reservoir recirculation conduit 346. Pump inlet conduit 344 joins a flow sensor 348 which in turn is fluidly connected to the motor/pump unit 298. A pump outlet conduit 350 connects the pump 298 to the pump flow director valve 302. The reservoir recirculation conduit 346 branches off of the pump outlet conduit 350. The pump flow director valve 302 is connected to a pump recirculation conduit 352 which in turn joins the pump inlet conduit 344. The pump flow director valve 302 is further connected to a distributor conduit 354. The distributor conduit joins a catheter branch 356, a balloon branch 358 and a valve branch 360. The branch lines 356, 358 and 360 connect to the catheter valve 304, the balloon valve 306 and the waste valve 308, respectively. These three solenoid valves connect to the fitting 288 via catheter supply line 362, balloon supply line 364 and valve supply line 366, respectively. The fitting connects to the fluid tubing 216. Specifically, the fitting 288 provides fluid communication between: a) catheter supply line 362 and catheter lumen 386; b) balloon supply line 364 and balloon lumen 384; and c) valve supply line 366 and control valve lumen 388.

The use, operation and function of the irrigation device 210 and its hydraulic control circuit 340 are as follows. The following description is provided in reference to TAI. However, it will be understood that the same or similar process may be carried out for stoma irrigation. In preparation for use the user unpacks the irrigation device as shown in FIG. 11. The controller 222 is removed from its storage location in the collar 230 of the reservoir. The reservoir 214 is filled either by removing it from the pump base unit 212 and carrying it to a faucet, or by removing the funnel 234 from the reservoir and placing the funnel under a faucet with the fill tube extending from the funnel to the reservoir. Either way, the reservoir 214 is filled with warm tap water, at the appropriate temperature (between 36 °C and 38 °C) and placed back on the pump base unit 212 if need be. The rectal catheter 220, and more specifically its manifold 260, is placed in the slot 252 between the clevis arms 250 of the connector hub 218. Doing so places the passageways 268, 270, and 272 in fluid communication with the fluid tubing 216. Note that the pump base unit 212 is not powered on during this preparation phase.

The next step is to power up the wireless electronic controller 222 and the pump base unit 212. The power on LED 326 should light up. The user should check the battery level indicators 322, 324 to make sure sufficient battery power is available to carry out the procedure. In this connection it should be noted that the pump motor 296 is only turned on at its appropriate stage and for only the required duration so as to prevent back pressure, noise and unnecessary battery drain.

When the motor is running there must always be an open passageway through the hydraulic control circuit 340. After powering up, the controller 222 will perform a water temperature check and call out the temperature on the controller's numeric display 328, while lighting the letter C on the units indicator 330. In addition, the water temperature gauge 314 will show red if the temperature is too high, blue if the temperature is too low and green if the temperature is in the proper range. The gauge 314 will continuously provide an indication of the water temperature even after the user starts scrolling through the other functions, which will cause the numeric display 328 and units indicator 330 to depart from the temperature readout.

In the following description of the hydraulic control circuit 340, passageways that are closed by one of the solenoid valves 300-308 are shown with an X through them. In addition, any blocked passageways at a particular stage are shown in dotted lines to indicate that no flow is active in that passageway at the stage under consideration. Arrows indicate the direction of active flow.

### Stage 1: Priming Stage

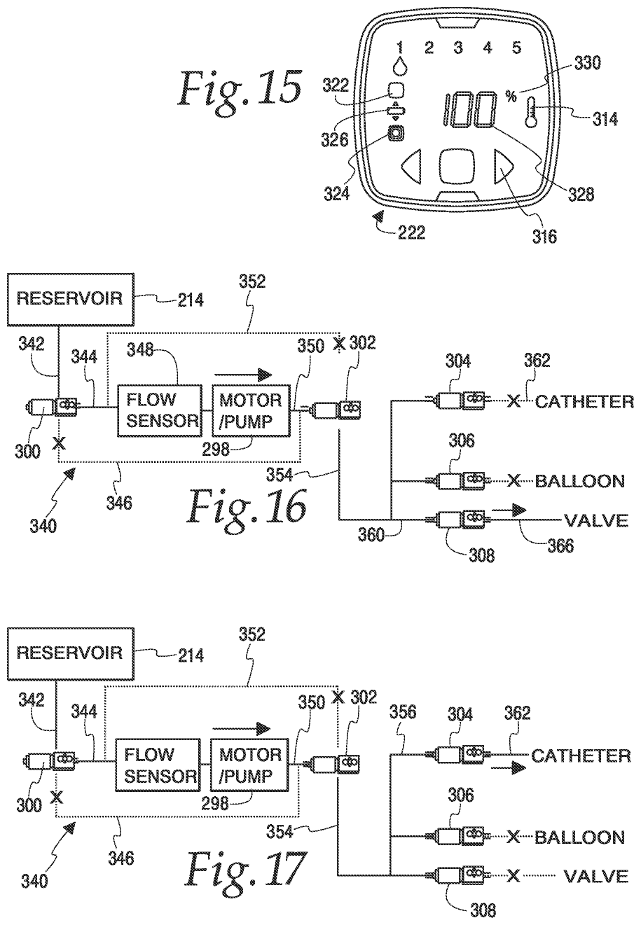

FIGS. 15-17 illustrate Stage 1 of the operation. Stage 1 is the priming stage. Before the catheter 220 is inserted into the rectum, the tubing needs to be primed in order to remove any air therein. That is, the catheter lumen 386 and the control valve lumen 388 of the fluid tubing 216 and their associated passageways are filled with water, as is the main passage 262 of the catheter. The volume of water to be pumped for priming will be pre-defined. Hence, the user will select the first icon on the wireless controller by pressing the forward button 316 once and the activate stage button 320 once. This will result in the Stage 1 icon to blink or flash until the priming is complete. In one example the numeric display 328 may read out the percentage of stage completion (0-100%) and the percentage icon of the units indicator 330 will light up as in FIG. 15. If the waste control valve 266 was not closed prior to the onset of Stage 1, it will be closed during the priming stage. For that matter, the waste control valve 266 is closed during all other stages except the evacuation Stage 4.

The first phase of Stage 1 is the priming of the waste control valve 266 and the associated passageways leading to it. For this phase the reservoir flow director valve 300 opens the reservoir conduit 342 and the pump inlet conduit 344 and closes the reservoir recirculation conduit 346 as shown in FIG. 16. The pump flow director valve 302 closes the pump recirculation conduit 352 and opens pump outlet conduit 350 and the distributor conduit 354. The catheter and balloon valves 304, 306 remain closed while the waste valve 308 is opened. This permits flow to the control valve lumen 388 of the fluid tubing 216 via the valve branch 360 and valve supply line 366. From the control valve lumen 388 water flows to the passageway 272 in the manifold 260. Once the passageway is filled the waste control valve 266 is closed and the pump motor 296 will turn off, completing the first phase of the priming Stage 1.

The second phase of the priming Stage 1 is shown in FIG. 17. The flow director valves 300, 302 and the balloon valve 306 remain set as before. But the waste valve 308 is closed and the catheter valve 304 is opened. The pump motor 296 is turned on and water flows to the catheter lumen 386 in the fluid tubing 216 via the catheter branch 356 and the catheter supply line 362. From the catheter lumen 386 water flows to the catheter passageway 270 in the manifold 260. The priming stage will also fill the passage 262 of the shaft 236 so that as much air as possible is expelled prior to insertion. Once these passageways are filled the pump motor 296 will turn off, completing the second phase of the priming Stage 1. Note that the motor will turn off before any irrigation fluid can exit the main passage 262 of the catheter shaft 236.

### Stage 2: Rectal Catheter Retention Balloon Inflation

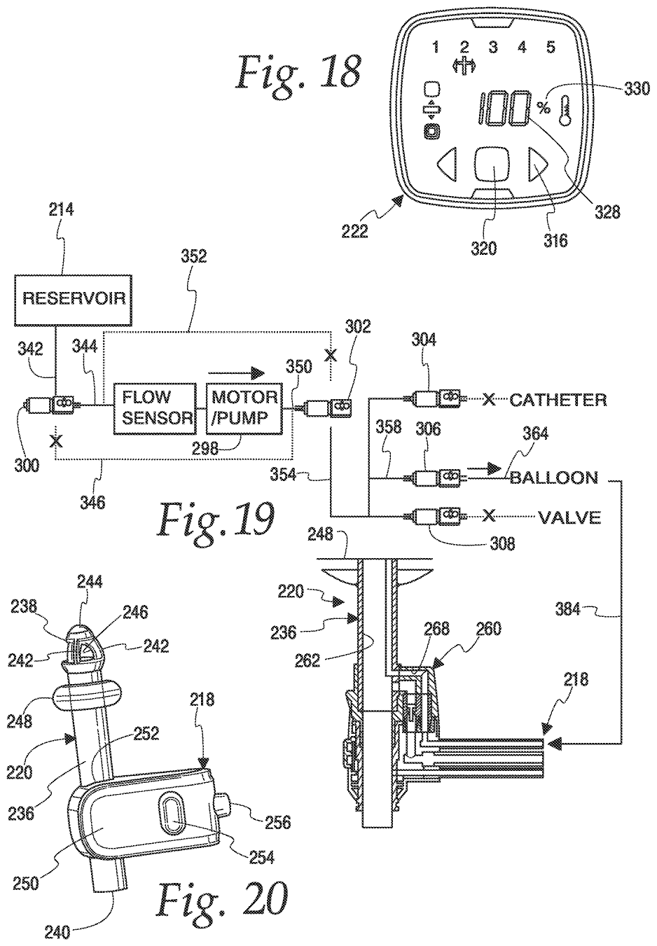

With the passageways in and to the catheter and waste control valve primed, the catheter 220 will be safely inserted into the rectum in accordance with the clinician's training. Stage 2 can then begin. This is the balloon inflation stage. The user will select the second icon on the wireless controller by pressing the forward button 316 once and the activate stage button 320 once. This will cause the Stage 2 icon to blink or flash until the balloon inflation is complete. The numeric display 328 may read out the percentage of how much of the total balloon volume is inflated (0-100%) and the percentage icon of the units indicator 330 may light up as in FIG. 18. The volume of water to be pumped into the retention balloon 248 will be pre-defined and will vary from user to user. The controller 222 has a programming mode in which the volume can be set.

A user may also adjust the balloon size during the TAI procedure if need be by using the forward and back buttons 316, 318, e.g. if leakage occurs after water is instilled into the rectum. For instance, the controller may be configured to adjust the balloon volume in increments as small as 10 mL. The controller may be configured to adjust in balloon volume in other increments without departing from the scope of the disclosure. The numeric display 328 could provide feedback to quantify the incremental increase/decrease through a percentage output. For example, the display 328 may display values of 25%, 50%, 75%, etc., to indicate the percentage amount of the total balloon size that is inflated. In an example, the maximum diameter for the retention balloon is 80 mm. In this instance, if the balloon was to be filled to 40 mm, the display 328 would display 50%. Additionally, the controller 222 includes a processor 317a and a memory 317b where adjusted inflation values may be stored. In instances where the inflation size of the balloon is adjusted from the pre-determined default size during Stage 2, the controller 222 is configured to remember/store the adjusted size for subsequent use. For example, processor 317a and memory 317b will remember/store the adjusted size for subsequent use so that during Stage 2 of a subsequent TAI procedure with device 210 the retention balloon 248 will be inflated to the adjusted size of the previous TAI procedure.

During balloon inflation the hydraulic control circuit is set as in FIG. 18. For this Stage 2 the reservoir flow director valve 300 opens the pump inlet conduit 344 and closes the reservoir recirculation conduit 346. The pump flow director valve 302 closes the pump recirculation conduit 352 and opens the distributor conduit 354. The catheter and waste valves 304, 308 remain closed while the balloon valve 306 is opened. This permits flow to the balloon lumen 384 of the fluid tubing 216 via the balloon branch 358, the balloon supply line 364 to the balloon lumen 384. From there water flows to the balloon passageway 268 and ultimately to the interior of the retention balloon 248. This results in the balloon volume increasing, as in FIG. 20, thus retaining the catheter inside the rectum.

### Stage 3: Transfer of Irrigant From the Reservoir Into the Rectum

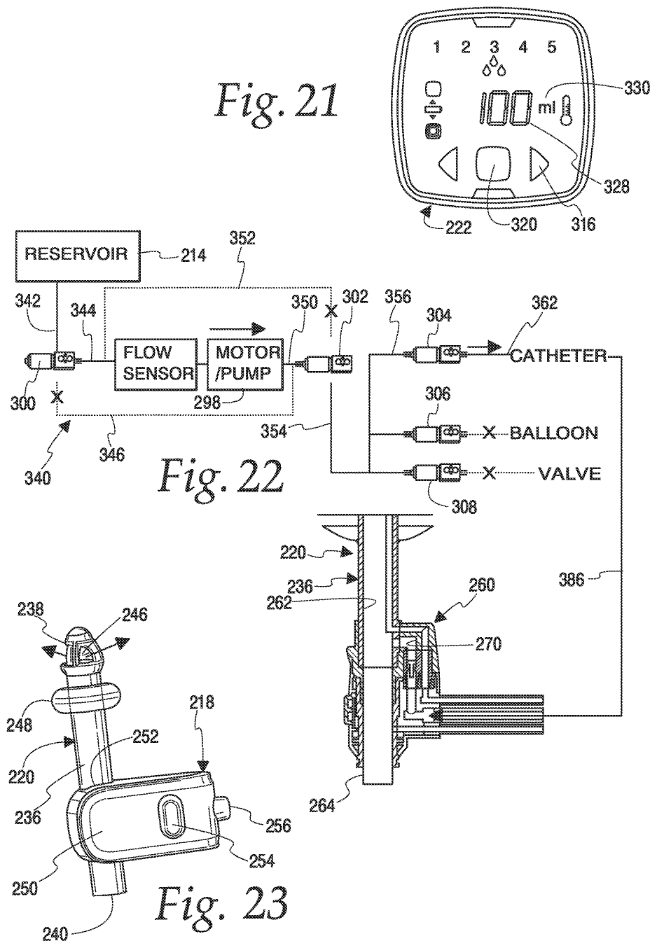

With the catheter 220 inserted and the balloon 248 inflated, the next stage can begin. This is Stage 3, the introduction of irrigation fluid (most commonly water) into the rectum. During this stage, the flow of liquid may be continuous or it may be a pulsatile flow by turning the pump motor 296 or pump flow director valve 302 on and off rapidly. The user will select the third icon on the wireless controller 222 by pressing the forward button 316 once and the activate stage button 320 at least once. In an example, the user can pre-program a set volume. Alternately, each time the user presses the activate stage button 100 ml of irrigant will be pumped. Pressing the activate stage button will cause the Stage 3 icon to blink or flash until the irrigant introduction is complete. The numeric display 328 will read out the milliliters of fluid pumped and the units indicator 330 will light up the letters “mL” as in FIG. 21.

The condition of the hydraulic control circuit during Stage 3 is shown in FIG. 22. The reservoir flow director valve 300 opens the pump inlet conduit 344 and closes the reservoir recirculation conduit 346. The pump flow director valve 302 closes the pump recirculation conduit 352 and opens the distributor conduit 354. The balloon and waste valves 306, 308 remain closed while the catheter valve 304 is opened. This permits flow to the catheter lumen 386 of the fluid tubing 216 via the catheter branch 356, the catheter supply line 362 to the catheter lumen 386. From there water flows to catheter passageway 270 in the manifold 260 and from there to the main passage 262 in the shaft 236 and ultimately out the apertures 246 to the rectum, as indicated in FIG. 23. Once the required amount of irrigant has been pumped the motor will turn off and the catheter valve 304 is closed. Note that the check valve 274 in the catheter passageway 270 in manifold 260 permits flow from the pump base unit 212 to the catheter 220, but prevents flow in the opposite direction. This prevents any fecal matter from contaminating anything in the connector hub 218 or the fluid tubing 216.

### Stage 4: Evacuation of Waste Stage

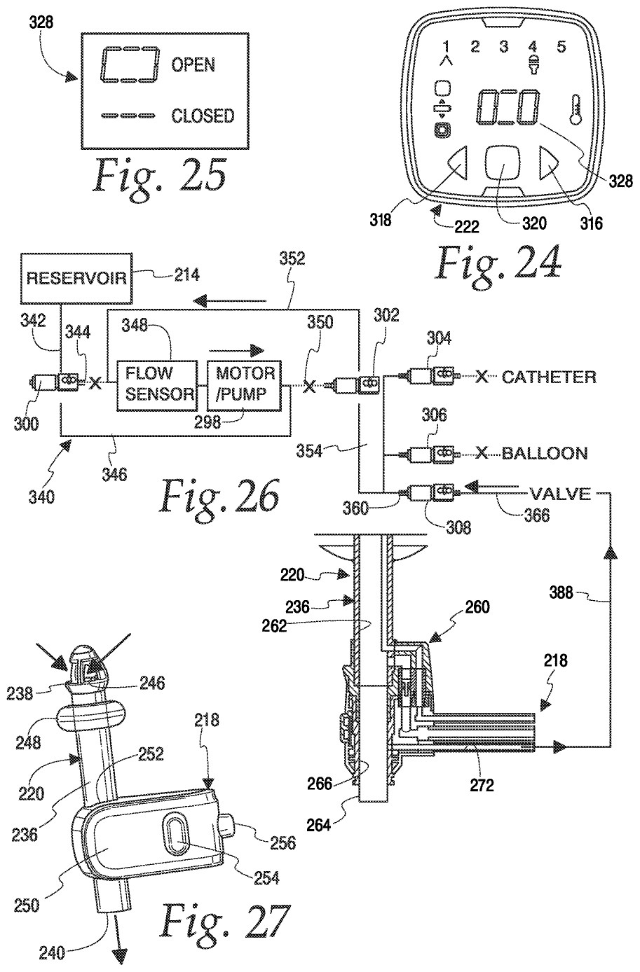

After the appropriate volume of water has been inserted into the rectum, it shall be allowed to irrigate the rectum for a defined period of time. Thereafter, the waste control valve 266 is opened to enable the liquified fecal matter to exit through the catheter shaft and into a toilet or a waste collection bag. This is Stage 4, the evacuation stage. The user will select the fourth icon on the wireless controller 222 by pressing the forward button 316 once and the activate stage button 320 once. Pressing the activate stage button will cause the Stage 4 icon to blink or flash while the waste control valve 266 is open and it will show continuously when the waste control valve is closed as shown in FIG. 24. Further, the numeric display 328 will read out the status of the waste control valve 266 as either open or closed, as shown in FIG. 25. The units indicator 330 will be shut off.

The condition of the hydraulic control circuit during Stage 4 is shown in FIG. 26. For the first time in the procedure the reservoir flow director valve 300 closes the pump inlet conduit 344 and opens the reservoir recirculation conduit 346. Also for the first time the pump flow director valve 302 opens the pump recirculation conduit 352 and closes pump outlet conduit 350. The distributor conduit 354 remains open. The catheter and balloon valves 304, 306 remain closed while the waste valve 308 is opened. The pump is turned on. This permits a reverse flow from the waste control valve 266 to the control valve passageway 272 in the manifold 260, to the control valve lumen 388 of the fluid tubing 216, to the valve supply line 366, through the waste valve 308, to the valve branch 360, to the distributor conduit 354, through the pump flow director valve 302, to the pump recirculation conduit 352, to the flow sensor 348, and to the pump 298. From there the pump directs water to the reservoir recirculation conduit 346, through the reservoir flow director valve 300 and reservoir conduit 342 and ultimately back to the reservoir 214. The result of all this is the waste control valve 266 opens. The pump is turned on long enough to return the same amount of liquid that was put into the waste control valve initially during the priming Stage 1. Once the waste control valve has been completely opened, the pump is turned off and the flow director valves are de-energized. With the waste control valve open, liquified fecal matter can enter the catheter shaft 236 through the apertures 246, as indicated by the arrows in FIG. 27. The fecal matter exits through the waste control valve and the bottom of the catheter shaft. It empties into a toilet or a waste collection bag (neither shown here).

Once the patient's rectum has been emptied of the liquified stool, the waste control valve 266 is closed by pressing the activate stage button once. During Stage 4 the activate stage button toggles the waste control valve 266 between the open and closed conditions. If the patient is not confident they have successfully evacuated all stool content, they can press the back button 318 once to select Stage 3. Pressing the activate stage button 320 at that point will start a new Stage 3, introducing irrigant into the rectum a second time. After the prescribed period of wait time, this is followed by another Stage 4 operation, as described above. This series of steps is repeated as needed.

### Stage 5: Rectal Catheter Retention Balloon Deflation

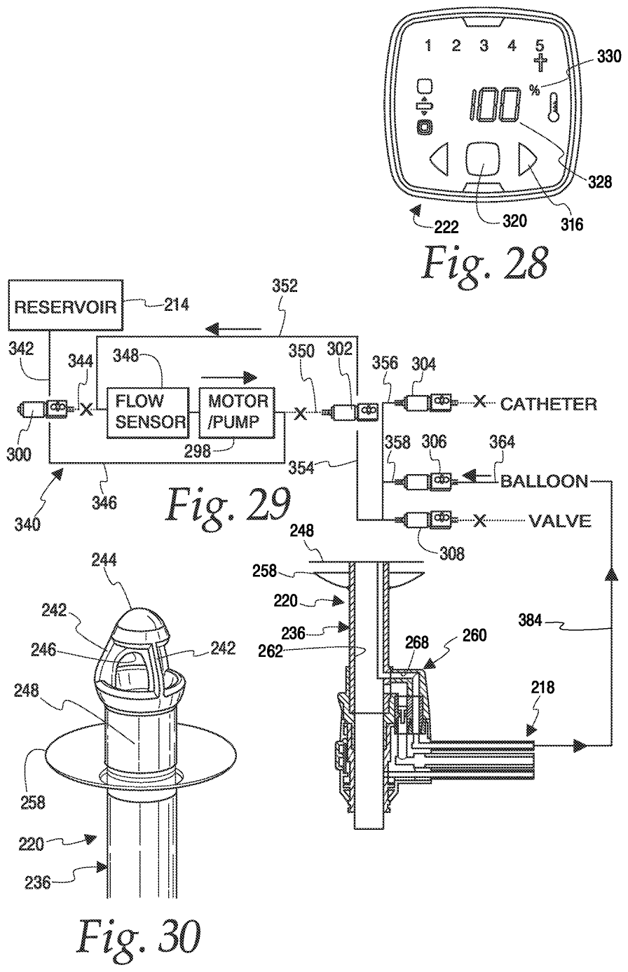

When the patient is confident that they have completely removed all stool, the catheter 220 needs to be removed from the rectum. To do this the retention balloon 248 must be deflated. The patient selects the Stage 5 icon by pressing the forward button 316 once to advance from the Stage 4 to the Stage 5deflation stage and then pressing the activate stage button 320 once. The numeric display 328 may read out the percentage of stage completion (0-100%) and again the percentage icon of the units indicator 330 may light up as in FIG. 28.

The condition of the hydraulic control circuit during Stage 5 is shown in FIG. 29. As in Stage 4, the reservoir flow director valve 300 closes the pump inlet conduit 344 and opens the reservoir recirculation conduit 346. Also, as in Stage 4, the pump flow director valve 302 opens the pump recirculation conduit 352 and closes pump outlet conduit 350. The distributor conduit 354 remains open. The catheter and waste control valves 304, 308 are closed while the balloon valve 306 is opened. The pump is turned on. This permits a reverse flow from the retention balloon 248 to the balloon passageway 268, to the balloon lumen 384 of the fluid tubing 216, to the balloon supply line 364, through the balloon valve 306, to the balloon branch 358, to the distributor conduit 354, through the pump flow director valve 302, to the pump recirculation conduit 352, to the flow sensor 348, and to the pump 298. From there the pump directs water to the reservoir recirculation conduit 346, through the reservoir flow director valve 300 and reservoir conduit 342 and ultimately back to the reservoir 214. The result of all this is the retention balloon 248 deflates. The pump 298 is turned on long enough to return the same amount of liquid that was put into the retention balloon initially during the inflation Stage 2. Once the retention balloon 248 is fully deflated as seen in FIG. 30, the pump is turned off and the flow director valves are de-energized. The user can then safely remove the catheter 220 from the rectum, disconnect the catheter 220 from the connector hub 218 and dispose of the catheter hygienically. The connector hub 218 and everything but the catheter and its manifold can be reused.

It can be seen from the foregoing description that all fluid passageways in the hydraulic control circuit 340 are independent of each other. There is no condition of the hydraulic control circuit 340 that permits the fluid passageways to communicate with each other. This ensures that water from the deflated catheter balloon 248 only returns to the water reservoir 214 and not into the catheter shaft 236 or other tubing lumens. In other words, the design does not permit balloon water to travel back to the catheter 220 and unnecessarily fill the rectum with the “left over” water from the balloon. Also, the presence of a waste control valve 266 permits multiple introductions of irrigation fluid if need be with only a single insertion of the catheter 220. Prior art catheters have to be inserted and removed once for each introduction of irrigation fluid. Thus, if multiple irrigant introductions are needed, multiple insertions and removals of the catheter are required in the prior art, presenting challenges in terms of hygiene and ease of use. The present disclosure removes this need for multiple insertions and removals.

It should be understood that components of the examples described herein and described in International Application Publication WO 2018/009871, which has been incorporated by reference herein in its entirety, are not mutually exclusive and may be used with other examples of irrigation devices.

It should be understood that various changes and modifications to the presently preferred embodiments described herein will be apparent to those skilled in the art. Such changes and modification can be made without departing from the spirit and scope of the invention disclosed herein.

1. An irrigation device, comprising:
a pump;
an irrigation fluid reservoir in fluid communication with the pump;
a rectal or stoma catheter in fluid communication with the pump and the irrigation fluid reservoir, wherein the rectal or stoma catheter has a retention balloon; and
a controller configured to:
inflate the retention balloon to a pre-determined default inflation size;
incrementally adjust the inflation size of the balloon from the pre-determined default inflation size; and
remember the adjusted balloon inflation size.

2. The irrigation device of claim 1, wherein the controller is configured to inflate the balloon to one of five pre-determined default inflation sizes.

3. The irrigation device of claim 2, wherein the first pre-determined default inflation size is 9 mm, the second pre-determined default inflation size is 27 mm, the third pre-determined default inflation size is 45mm, the fourth pre-determined default inflation size is 63 mm, and the fifth pre-determined default inflation size is 80 mm.

4. The irrigation device of claim 1, wherein the controller is configured to incrementally adjust the inflation size of the retention balloon from the pre-determined inflation size in at least 10 mL increments.

5. The irrigation device of claim 1, wherein the controller comprises a first button to increase the inflation size of the retention balloon and a second button to decrease the inflation size of the retention balloon.

6. The irrigation device of claim 1, wherein the controller comprises a display, wherein the display provides a visualization of the adjusted inflation size of the retention balloon.

7. The irrigation device of claim 6, wherein the display provides the adjusted inflation size of the retention balloon as a decimal number.

8. The irrigation device of claim 6, wherein the display provides the adjusted inflation size of the balloon as a percentage of the total balloon size that is inflated.

9. The irrigation device of claim 1, wherein the controller is configured to automatically inflate the retention balloon to the remembered adjusted inflation size.

10. The irrigation device of claim 1, wherein the controller is a wireless controller.

11. A method for trans-anal or stoma irrigation comprising:
inserting a catheter of an irrigation device into a rectum or stoma, wherein the catheter has a retention balloon, and wherein the irrigation device includes:
a pump in fluid communication with the catheter;
an irrigation fluid reservoir in fluid communication with the pump and catheter, and
a controller, wherein the controller is configured to:
inflate the retention balloon to a pre-determined default inflation size;
incrementally adjust the inflation size of the retention balloon from the pre-determined default inflation size; and
remember the adjusted inflation size of the retention balloon;
inflating the retention balloon to the pre-determined default inflation size;
adjusting the inflation size of the retention balloon from the pre-determined default inflation size;
irrigating the rectum or stoma;
deflating the retention balloon; and
removing the catheter.

12. The method of claim 11, further comprising automatically inflating the retention balloon to the adjusted inflation size during a subsequent trans-anal or stoma irrigation procedure.

13. The method of claim 11, wherein the controller is configured to inflate the retention balloon to one of five pre-determined default inflation sizes.

14. The method of claim 13, wherein the first pre-determined default inflation size is 9 mm, the second pre-determined default inflation size is 27 mm, the third pre-determined default inflation size is 45 mm, the fourth pre-determined default inflation size is 63 mm, and the fifth pre-determined default inflation size is 80 mm.

15. The method of claim 11, wherein the controller is configured to incrementally adjust the inflation size of the retention balloon from the pre-determined default inflation size in at least 10 mL increments.

16. The method of claim 11, wherein the controller comprises a first button to increase the inflation size of the retention balloon and a second button to decrease the inflation size of the retention balloon.

17. The method of claim 11, wherein the controller comprises a display, wherein the display provides a visualization of the adjustment made to the inflation size of the retention balloon.

18. The method of claim 17, wherein the display provides the adjusted inflation size of the retention balloon as a decimal number.

19. The method of claim 17, wherein the display provides the adjusted inflation size of the retention balloon as a percentage of the total retention balloon size that is inflated.

20. The method of claim 11, wherein the controller is a wireless controller.
